(19) United States
(12) Patent Application Publication
(10) Pub. No.: US 2014/0242612 A1
(43) Pub. Date: Aug. 28, 2014

(54) SYSTEM AND METHOD FOR INTEGRATION OF MOBILE DEVICE IMAGING WITH MICROCHIP ELISA

(71) Applicants: Shuqi Wang, Medford, MA (US); Utkan Demirci, Cambridge, MA (US); Bin Ye, Brookline, MA (US); Ragip Akbas, Nashua, NH (US)

(72) Inventors: Shuqi Wang, Medford, MA (US); Utkan Demirci, Cambridge, MA (US); Bin Ye, Brookline, MA (US); Ragip Akbas, Nashua, NH (US)

(21) Appl. No.: 14/131,853

(22) PCT Filed: Jul. 16, 2012
(86) PCT No.: PCT/US2012/046951

§ 371 Date: May 5, 2014

Related U.S. Application Data
(60) Provisional application No. 61/507,751, filed on Jul. 14, 2011.
(60) Provisional application No. 61/515,127, filed on Aug. 4, 2011.

(51) Int. Cl. G01N 33/543; G01N 33/569; G01N 33/574

(52) U.S. Cl. CPC G01N 33/54386; G01N 33/57449; G01N 33/56916; G01N 33/56972
USPC 435/7.23; 435/287.2; 435/7.92; 435/7.37; 435/7.24

(57) ABSTRACT

A system and method for analyzing a biomarker in a biological sample is provided. A biological sample is loaded onto a microchip and an enzyme-linked immunosorbent assay specific to the biomarker is performed on the microchip. A color image of the microchip is generated using a mobile device and a color intensity of a selected portion of the color image is determined. The color intensity is correlated with a biomarker concentration using a baseline curve calculation and the concentration of the biomarker is then reported.

### CROSS-REFERENCE TO RELATED APPLICATIONS

This application is based on, claims the benefit of, and incorporates herein by reference, U.S. Provisional Patent Application Ser. No. 61/507,751 filed on Jul. 14, 2011, and entitled “Integration of Cell Phone Imaging with Microchip ELISA to Detect Ovarian Cancer HE4 Biomarker from Urine at the Point-of-Care,” and U.S. Provisional Patent Application Ser. No. 61/515,127 filed on Aug. 4, 2011, and entitled “Microfluidics-Based Apparatus and Systems and Methods of Using the Same.”

### STATEMENT REGARDING FEDERALLY SPONSORED RESEARCH

This invention was made with government support under DAMD17-02-2-0006, W81XWH-07-2-0011, and W81XWH-09-2-0001 awarded by U.S. Army Medical Acquisition Activity (USAMRAA) and under grant number NIH U01 HL065899-08, awarded by the National Institutes of Health. The government has certain rights in the invention.

### BACKGROUND OF THE INVENTION

The field of the invention is systems and methods for non-invasive point-of-care biomarker detection. More particularly, the invention relates to systems and methods for detecting cancer biomarker concentrations in a biological sample, such as urine, using microchip enzyme-linked immunoassays (ELISAs) and a mobile device or lensless charge coupled-device for imaging the microchip ELISAs and analyzing the images to determine cancer biomarker concentrations.

Cancer detection and treatment is a substantial component in the practice of modern medicine. For example, ovarian cancer is the fifth leading cause of all cancer related mortality among women. Since ovarian cancer is asymptomatic at early stages, most patients present with advanced disease (such as stage III or stage IV) when diagnosed. Despite radical surgery and chemotherapy, the five-year survival rate of ovarian cancer at stages III and IV is only 33% compared to 90% at stage I. This statistic alone highlights the need for early diagnosis and large scale screening, at least among high-risk populations. However, existing diagnosis methods such as biopsy, medical imaging, and genetic analysis cannot be used frequently for routine screening, and oftentimes lengthy and complex testing procedures associated with these methods hinder high-risk populations from seeking immediate medical care. Thus, the lack of cost-effective methods that can achieve frequent, simple and non-invasive testing hinders early detection and renders high mortality in ovarian cancer patients.

Annual transvaginal sonography has been used to screen for ovarian cancer among subjects with a family history of ovarian cancer, which has shown limited efficacy when the ovarian volume remains normal. Another common screening method is a serum CA125 test, an enzyme-linked immunosorbent assay (ELISA) with a sensitivity of 72% at specificity 95%. The sonography and serum screening methods are invasive, costly, and provide results that are instrument dependent and, as a result, they cannot be reasonably established at point-of-care (POC) settings.

POC diagnostics are appealing in terms of disease monitoring and control, including infectious diseases, cancer and diabetes, in both resource-limited and resource-rich settings. To offer POC testing by the bedside, the World Health Organization (WHO) has expressed the need for inexpensive, disposable, and easy-to-use diagnostic devices, for example for resource-limited settings where there are limitations with trained personnel, infrastructure, and medical instruments. Features of such devices should include functionality under high humidity and temperature, and robust operation in the absence of reliable electricity and water supply. The need for such devices also extends to resource-rich settings such as airports, community clinics, and emergency rooms, where frequent testing and rapid results are needed, or access to central laboratories may be limited (for example, for blood sugar testing or influenza screening).

With advances in microelectromechanical systems (MEMS), miniaturization of ELISA on a single microchip has become feasible. Microchip ELISA results can be seen by the naked eye; however, analyte concentrations cannot be quantitatively measured using this method. Quantitative detection technologies such as fluorescence detection, chemiluminescence or electrical detection are expensive, technologically complex, and require bulky detection setups. For instance, fluorescence or chemiluminescence detection often requires the use of a charge-coupled device (CCD) camera interfaced with an expensive fluorescence microscope. Electrical detection of microchip ELISA requires a reliable power supply and delicate circuitry to measure the change in impedance induced by the analyte. Colorimetric detection of on-chip ELISA requires a CCD camera coupled to a microscope and connected to a computer with an analysis program. Thus, all of these solutions require a laboratory environment to be utilized. Thus, despite the widespread need, current state-of-the-art diagnostic technologies such as polymerase chain reaction (PCR), ELISA, or microarray have practical challenges hindering them from being established at the POC. Simply, as described above, these detection methods are not ideal for POC testing despite the use of miniaturized microchips and, thus, have not been adopted in for POC applications.

Considering the above, there is a need for an inexpensive, simple, and quick detection method and system to facilitate POC testing for cancer screening or detection.

### SUMMARY OF THE INVENTION

The present invention overcomes the aforementioned drawbacks by providing a system and method for detecting microchip ELISA results using a mobile device with an imaging apparatus to measure a biomarker, cell, or pathogen (such as virus or bacteria) concentration in clinical samples. For example, the mobile device may have an integrated mobile application or a lensless charge-coupled device connected to an additional device with an integrated application, thereby facilitating point-of-care testing. A biological sample, such as urine, is loaded into a microchip system configured to provide colorimetric biomarker feedback. The colorimetric feedback is imaged by the mobile device and analyzed using the mobile device, either directly using the processing systems of the mobile device or through communication with a remote processing system using the communications systems of the mobile device, to provide point-of-care (POC) testing results.

It is an aspect of the invention to provide a system for point-of-care (POC) testing of biological samples for biomarkers indicative of a predetermined pathological condition. The system includes a microchip system configured to receive a biological sample secured from a patient and provide colorimetric biomarker feedback indicative of a testing related to the predetermined pathological condition. The system also includes a mobile device configured to access a communications network and having a processor configured to access a camera configured to acquire color images of the colorimetric biomarker feedback and determine a color intensity of at least a selected portion of the color image. The processor is also configured to correlate the color intensity of the selected portion of the color image with a biomarker concentration and generate a report regarding the concentration of the biomarker concentration.

It is an aspect of the invention to provide a mobile-device based method for analyzing a biomarker in a biological sample. The method includes loading the biological sample onto a microchip, performing an enzyme-linked immunosorbent assay specific to the biomarker on the microchip, and generating a color image of the microchip using one of a mobile device and a lensless charge-coupled device. The method also includes determining a color intensity of a selected portion of the color image, correlating the color intensity with a biomarker concentration using a baseline curve calculation, and reporting the concentration of the biomarker.

It is another aspect of the invention to provide a charge-coupled device-based method for analyzing a biomarker in a biological sample. The method includes loading the biological sample onto a microchip, performing an enzyme-linked immunosorbent assay specific to the biomarker on the microchip; generating a color image of the microchip using a lensless charge-coupled device, and transmitting the color image to an additional device. The steps of the method also includes determining a color intensity of a selected portion of the color image, correlating the color intensity with a biomarker concentration using a baseline curve calculation, and reporting the concentration of the biomarker.

It is another aspect of the invention to provide a portable test system for mobile device camera-based analysis of biomarker concentrations in biological samples applied to a microchip. The system includes an enclosure adapted to receive the microchip and including an imaging aperture large enough to allow imaging of the microchip through the imaging aperture using the mobile device camera. The system also includes at least one light source adapted to illuminate the microchip and a power source adapted to power the at least one light source.

The foregoing and other aspects and advantages of the invention will appear from the following description. In the description, reference is made to the accompanying drawings which form a part hereof, and in which there is shown by way of illustration a preferred embodiment of the invention. Such embodiment does not necessarily represent the full scope of the invention, however, and reference is made therefore to the claims and herein for interpreting the scope of the invention.

### BRIEF DESCRIPTION OF THE DRAWINGS

FIGS. 1A and 1B are schematic representations of test systems for use with a mobile device and a lensless charge-coupled device, respectively, in accordance with embodiments of the present invention.

FIG. 2 is a flowchart setting forth the steps of an example of a method for mobile device camera-based or charge-coupled device-based colorimetric measurement of microchip ELISA results in accordance with some embodiments of the invention.

FIG. 3 is a pictorial representation of a microchip for use with practicing embodiments of the present invention.

FIG. 4 is a pictorial representation of an on-chip enzyme-linked immunosorbent assay (ELISA).

FIG. 5 is a pictorial representation of a mobile device imaging a microchip ELISA in accordance with embodiments of the present invention.

FIG. 6 is a pictorial representation of a mobile device display in accordance with embodiments of the present invention.

FIG. 7 is a pictorial representation of a microchip for use with embodiments of the present invention.

FIG. 8 is a series of graphs showing baseline curves correlating ovarian cancer biomarker human epididymis protein 4 (HE4) concentrations to variables measured from a completed ELISA, where FIG. 8A illustrates HE4 concentrations at given optical density measurements measured from a spectrophotometer, FIG. 8B illustrates HE4 concentrations at given measured pixel values from images captured by a mobile device, and FIG. 8C illustrates HE4 concentrations at given measured pixel values from images captured by a lensless charge-coupled device.

FIG. 9 is a series of bar plots comparing logarithmic HE4 concentrations from clinical samples of normal and cancer patients as determined through a conventional microplate ELISA method (FIG. 9A), a mobile device-based microchip ELISA method (FIG. 9B), and a charge-coupled device-based microchip ELISA method (FIG. 9C).

FIG. 10 is a series of box-whisker plots comparing logarithmic HE4 concentrations from clinical samples of normal and cancer patients as determined through a conventional microplate ELISA method (FIG. 10A), a mobile device-based microchip ELISA method (FIG. 10B), and a charge-coupled device-based microchip ELISA method (FIG. 10C).

FIG. 11 is series of Bland-Altman plots comparing HE4 concentrations determined by a conventional microplate ELISA method and a mobile device-based microchip ELISA method for clinical samples of cancer patients (FIG. 11A) and normal patients (FIG. 11B), and comparing HE4 concentrations determined by the conventional microplate ELISA method and a charge-coupled device-based microchip ELISA method for clinical samples of cancer patients (FIG. 11C) and normal patients (FIG. 11D).

FIG. 12 is a graph of receiver operating characteristic (ROC) analyses illustrating prediction accuracies for determining HE4 concentrations through a conventional microplate ELISA method, a mobile device-based microchip ELISA method, and a charge-coupled device-based microchip ELISA method.

FIG. 13 is a pictorial representation of a micro-a-fluidic ELISA, in accordance with some aspects of the invention.

FIG. 14 is an exploded-view pictorial representation of a micro-a-fluidic ELISA.

FIG. 15 is a schematic illustration of a micro-a-fluidic ELISA procedure.

FIG. 16 is a series of graphs showing standard curves correlating BDNF concentrations to variables measured from a completed ELISA, where FIG. 16A illustrates BDNF concentrations at given optical density measurements measured from a spectrophotometer of a microplate ELISA and FIG. 16B illustrates BDNF concentrations at given measured pixel values from images captured by a mobile device of a micro-a-fluidic ELISA.

FIG. 17 is a series of graphs showing standard curves correlating KIM-1 concentrations to measured pixel values from images, captured by a mobile device, of a completed micro-a-fluidic ELISA, where FIG. 17A illustrates KIM-1 concentrations at given measured pixel values for a thirty-minute micro-a-fluidic ELISA procedure and

FIG. 17B illustrates KIM-1 concentrations at given measured pixel values for a ten-minute micro-a-fluidic ELISA procedure.

FIG. 18 is a graph showing a standard curve correlating NGAL concentrations to measured pixel values from images, captured by a mobile device, of a completed micro-a-fluidic ELISA.

FIG. 19 is series of graphs related to micro-a-fluidic ELISA-based CD4 cell count detection, where FIG. 19A illustrates a standard curve correlating CD4 counts to measured pixel values from images, captured by a mobile device, of a completed micro-a-fluidic ELISA, FIG. 19B is a chart correlating CD4 counts determined by conventional flow cytometry and a mobile device-based micro-a-fluidic ELISA method for clinical samples of AIDS patients, and FIG. 19C is a Bland-Altman plot comparing CD4 counts determined by conventional flow cytometry and a mobile device-based micro-a-fluidic ELISA method for clinical samples of AIDS patients.

FIG. 20 is a series of graphs showing standard curve correlating E. coli concentrations to measured pixel values from images, captured by a mobile device, of a completed micro-a-fluidic ELISA, where FIGS. 20A and 20B illustrate E. coli concentrations given measured pixel values based on E. coli samples and FIG. 20C illustrates E. coli concentrations given measured pixel values based on blood samples spiked with E. coli.

FIG. 21 is a series of dot plots showing fluorescent-activated cell counting (FACS) data, where FIG. 21A illustrates a plot correlating forward scatter (FSC-H) and side scatter (SSC-H) and FIG. 21B illustrates a plot correlating specific fluorescent colors measured (FL1-H and FL2-H).

FIG. 22 is a series of graphs showing standard curves correlating neutrophil cell counts based on microchip methods to neutrophil cell counts based on conventionally FACS methods, where FIG. 22A illustrates a plot of all sample cell count results and FIG. 22B illustrates a plot of averaged sample cell count results.

### DETAILED DESCRIPTION OF THE INVENTION

The present invention provides a non-invasive ovarian cancer detection method that combines microchip enzyme-linked immunosorbent assay (ELISA) and mobile device camera-based or charge-coupled device (CCD)-based colorimetric measurement to detect biomarkers in a point-of-care testing system that can be implemented in physician offices in primary care or bedside settings. In accordance with the present invention, a mobile device integrated with a biomarker detection application enables immediate data processing of microchip ELISA results and reporting of biomarker concentrations without referring to peripheral equipment for read-out and analysis, thus facilitating point-of-care (POC) testing.

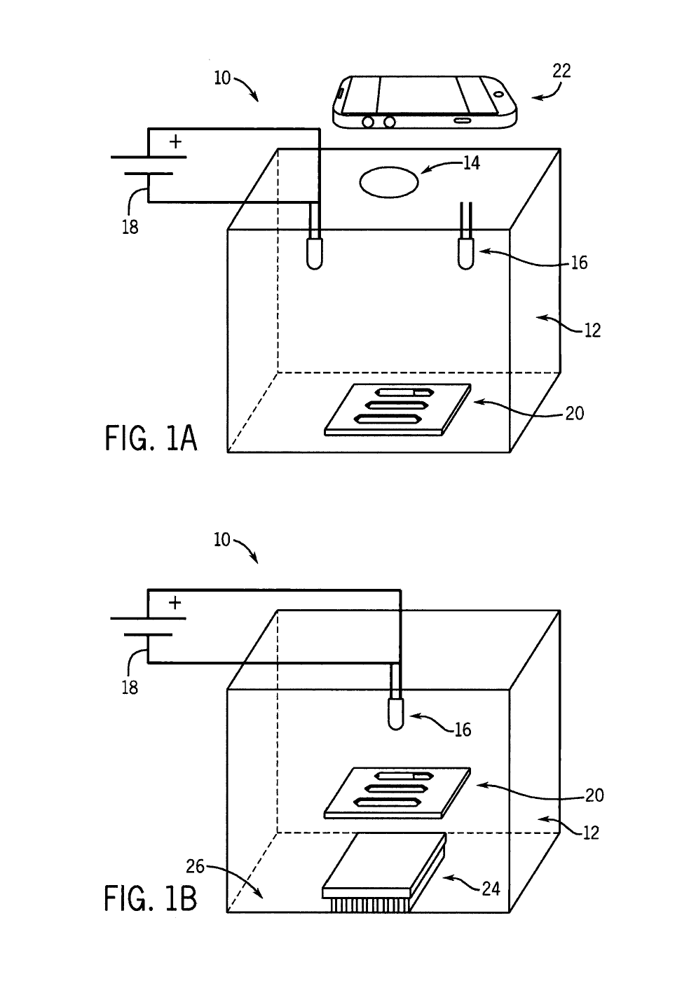

Referring to FIG. 1A, a test system 10 utilized for mobile device-based biomarker concentration analysis is illustrated. The test system 10 includes an enclosure 12 with an imaging port or aperture 14, one or more light sources 16, and a power source 18 for operating the light sources 16. A microchip ELISA 20, completed with a patient urine sample, can be positioned within the enclosure 12 opposite from the imaging port 14. A mobile device 22 (such as a smart phone, personal digital assistant, tablet computer, etc.) can be positioned over the imaging port 14 so that a built-in camera of the mobile device 22 can capture an image of the microchip ELISA 20 through the imaging port 14. As further discussed below, the obtained image can then be analyzed by an integrated mobile application on the mobile device 22. The application can differentiate the color intensity developed on the microchip, as a result of the completed ELISA, correlate the color intensity with a concentration of biomarkers in the biological samples, and report or display the concentration of the biomarker or a correlated predetermined condition, such as the presence or absence of cancer, to a user.

The test system of FIG. 1A provides standardized lighting for imaging the microchip ELISA 20 as well as a standardized distance between the microchip and the mobile device 22. In addition, the enclosure 12 and the light sources 16 provide sufficient control of the ambient lighting during imaging of the microchip ELISA 20 and block external light sources that may interfere with the imaging. In one implementation, the light sources 16 are white light emitting diodes (LEDs) and the power source 18 operating the white LEDs includes one or more batteries providing an approximate 3-volt source voltage. The enclosure 12 can be substantially small, where the distance between the imaging port 14 and the positioned microchip ELISA 20 is about 8 centimeters. Due to the small size of the enclosure 12, the use of batteries for powering the light sources 16, and the use of a mobile device 22 for imaging the microchip ELISA results, the test system 10 can be easily portable for use in POC environments. Furthermore, use of the integrated mobile application eliminates the need to export the image to another device for data processing to retrieve results.

Referring to FIG. 1B, a test system 10 utilized for CCD-based biomarker concentration analysis is illustrated. The test system 10 of FIG. 1B includes an enclosure 12, a light source 16, a power source 18 for operating the light sources 16, and a CCD 24. A microchip ELISA 20, completed with a biological sample, can be positioned within the enclosure 12 between the light source 16 and the CCD 24. For example, the CCD 24 may be placed within an indentation (not shown) in a wall 26 of the enclosure 12 and the microchip ELISA 20 is placed on the wall 26 over the indentation. In another example, the enclosure 12 includes a microchip holder (not shown) positioned to hold the microchip ELISA 20 directly over the CCD 24. The CCD is a lensless, color CCD capable of capturing a color image, or colorimetric readout, of the microchip ELISA 20. The CCD 24 is connected to a mobile device or an additional device, such as a laptop or a personal computer, including a light detector and an application for interpreting the color image to differentiate the color intensity developed on the microchip, as a result of the completed ELISA, and correlate the color intensity with a concentration of the biomarker in the biological sample. The CCD 24 can be connected to the mobile device or the additional device through wired connections or wireless connections (such as Bluetooth® or Wi-Fi).

Similar to the test system 10 of FIG. 1A, the test system 10 of FIG. 1B can provide standardized lighting for imaging the microchip ELISA 20 as well as a standardized distance between the microchip, the light sources 16, and the CCD 24. In addition, the enclosure 12 and the light source 16 can provide sufficient control of the ambient lighting during imaging of the microchip ELISA 20 and block external light sources that may interfere with the imaging. In one implementation, the light source 16 is a white light emitting diode (LED) and the power source 18 operating the white LED includes one or more batteries providing an approximate 3-volt source voltage. The enclosure 12 can be substantially small, where the distance between the light source 16 and the positioned microchip ELISA 20 is about 23 centimeters.

As described above, the CCD 24 of the present invention is a lensless detector. Conventional CCD-based imaging systems use CCDs coupled to lenses as part of an imaging apparatus, such as a confocal or fluorescence microscope. These systems are not suitable for POC testing because of the high cost, maintenance, and portability issues of the imaging apparatuses. In comparison, the lensless CCD test system 10 of the present invention is capable of detecting color changes without using a fluorescence microscope, therefore making the test system 10 more affordable, portable, and easier to maintain, facilitating its use in POC environments. Further, the lensless CCD 24 used with the test device 10 of the present invention has a wide field of view (FOV), which is significantly larger than that of a microscope and can immediately capture the whole microchip area without scanning (as scanners are also not desirable for resource-limited settings due to the cost and difficulty of maintenance). It is noted, however, that the following methods can be carried out using a CCD with a lens and/or color filters.

There are various biomarkers for cancer detection. In accordance with the present invention, one useful biomarker is human epididymis protein 4 (HE4), which can be used as a biomarker for ovarian cancer detection. Another useful biomarker is cancer-antigen 125 (CA125). Such biomarker concentrations in serum can be correlated with the clinical status of ovarian cancer. In addition, HE4 can be reliably detected in urine from ovarian cancer patients at both early (I/II) or late stages (III/IV). Also, urine is an easily secured biological sample. Accordingly, one desirable biomarker in accordance with the present invention is HE4 because biological samples can be readily secured at a POC and useful results regarding a predetermined condition that is highly useful clinically, the presence or absence of ovarian cancer, can be determined.

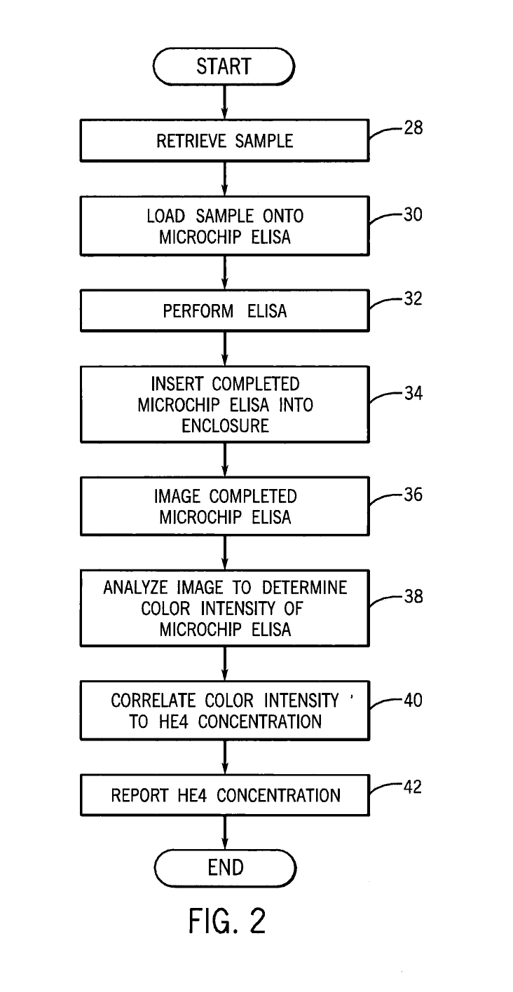

According to a method of the present invention as shown in FIG. 2, a user can retrieve a patient's urine sample (process block 28), load the urine sample onto a microchip (process block 30), perform an ELISA on the microchip to isolate and detect HE4, or another biomarker, in the biological sample (process block 32), insert the microchip with the completed ELISA into the enclosure (process block 34), image the microchip to generate a color image using a mobile device camera or a lensless CCD (process block 36), analyze the color image to determine the color intensity developed by the completed ELISA (process block 38), correlate the color intensity with a concentration of HE4 in the urine sample (process block 40), and report the HE4 concentration (process block 42). It is noted that the above method can be incorporated for on-chip ELISA to effectively detect other biomarkers or analytes of interest in biological samples other than urine (such as saliva, whole blood, serum, or plasma), as further discussed below. Furthermore, multiple biomarkers may be tested simultaneously on the same microchip (for example, each in different microchannels).

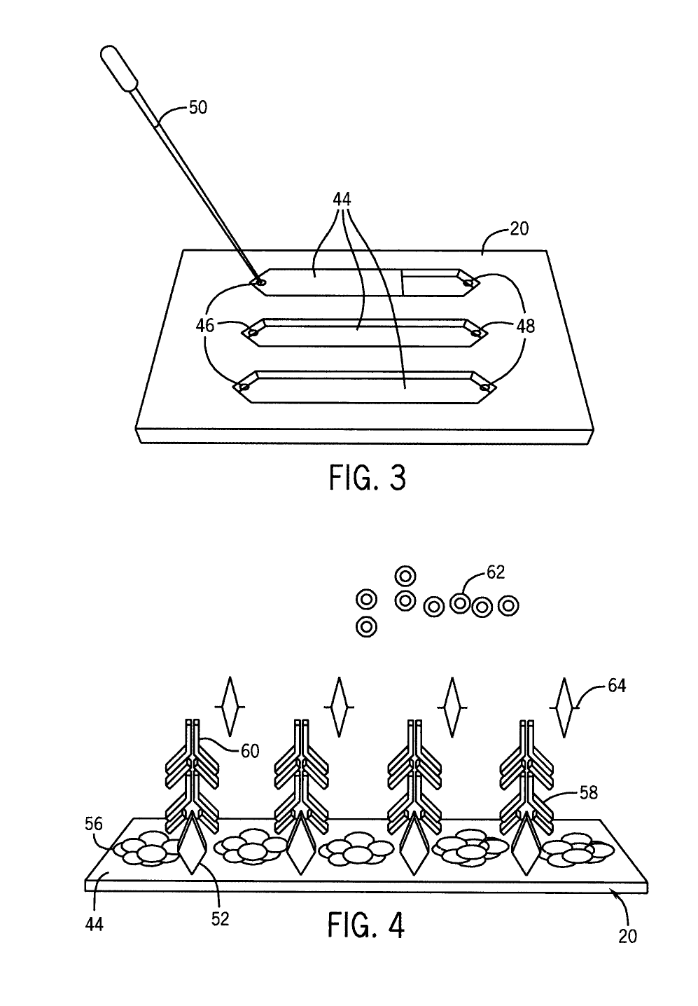

With reference to process block 30, a urine sample (for example, about 100 micro liters) can be loaded onto the microchip. This may be accomplished through manual pipetting, as shown in FIG. 3, or automated pipetting through the use of a micropump. As shown in FIG. 3, an example microplate ELISA 20 can include microchannels 44, each including an inlet 46 and an outlet 48. The urine sample can be loaded through one or all of the inlets 46 using a pipette 50. In some applications, non-lithographic techniques can be used to fabricate microchips for use with the present invention. In one implementation, polymethyl-methacrylate (PMMA) microfluidic chips are used.

With reference to process block 32, microchip ELISA is performed to isolate and detect the protein biomarker HE4 in the urine sample. The microchips (such as microchip ELISA 20 of FIGS. 1A, 1B, and 2) can be used to perform direct, indirect, or sandwich ELISA. For example, as shown in the example illustration of FIG. 4, once HE4 52 is captured by physical adsorption on the surface of the microchip 20, a blocking substrate, such as bovine serum albumin (BSA) 56 is applied. A detection antibody 58 is added and then an enzyme conjugated secondary antibody against HE4, such as a horseradish peroxidase (HRP) conjugated secondary antibody 60, is then added, forming an immuno-complex. Upon addition of a substrate, such as tetramethylbenzidine (TMB) 62, the enzyme 64 (of the enzyme conjugated secondary antibody 60) will catalyze the substrate 62, and initiate color development (for example, blue color development when using TMB 62). Application of these reagents can be performed through manual or automated pipetting. In addition, incubation may also be required between application of reagents. In some implementations, a capture antibody is added prior to sample injection and a sandwich microchip ELISA is performed, where HE4 52 is captured on the microchip 20 by the capture antibody.

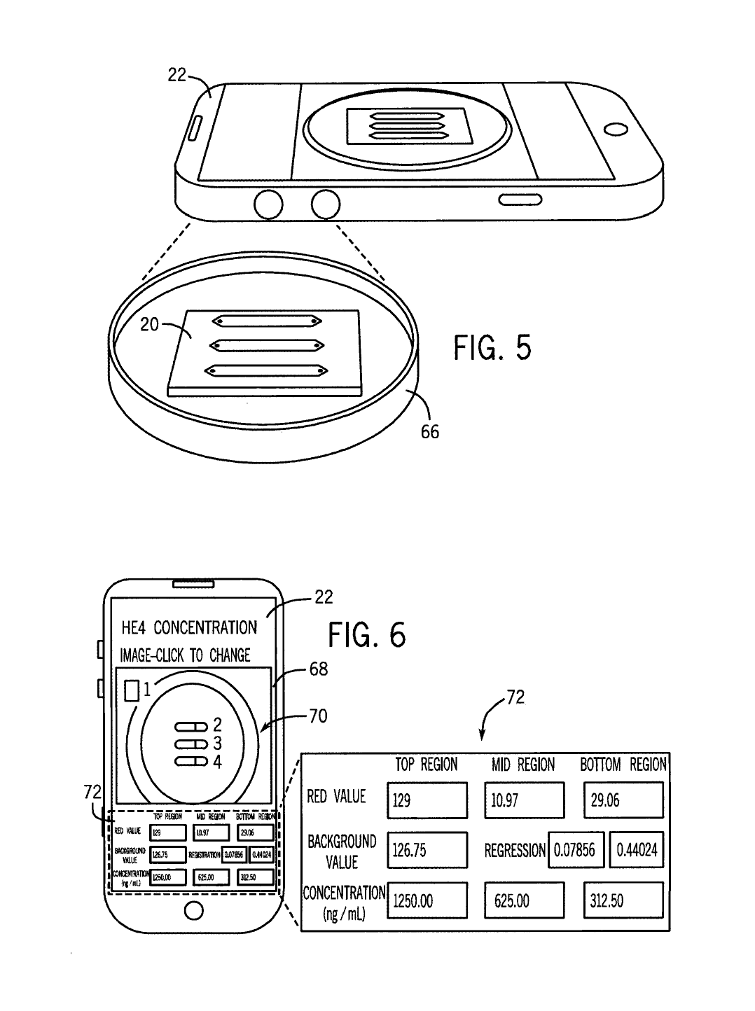

With reference to process block 34, the microchip ELISA is imaged using the mobile device (such as mobile device 22 of FIG. 1A) or a lensless CCD (such as CCD 26 of FIG. 1B). In accordance with process block 32, the microchip is first positioned within an enclosure and then imaged using the mobile device or the CCD (for example, as shown in FIGS. 1A and 1B). In some implementations, the microchip ELISA may be imaged outside of an enclosure. For example, as shown in FIG. 5, a mobile device 22 is used to image a microchip ELISA 20 on a Petri dish 66.

With reference to process blocks 38 and 40, image analysis and color intensity correlation can be performed by an application stored on and executed by a processor of the mobile device (such as an integrated mobile “app”) or the additional device connected to the CCD (such as an image processing software application). In other implementations, images may be transmitted to another device interface with patient medical records or a central database, for example, by way of a communications network connection provided by the mobile device or another device and process blocks 38 and 40 can be performed by an application stored on and executed by the other device. For example, images taken by the mobile device can be sent via a mobile network to the additional device for image analysis and color intensity correlation.

The application, executed by any of the above-described devices, can retrieve the color image of the microchip ELISA. For example, through the mobile device a user can execute the application and select an image for analysis on a home screen display of the application. In some instances, the application can provide the user with an option to create a new image, using the mobile device or the CCD, or choose a previously saved image. The application then processes the selected image by determining “detection regions” within the image that represent the microchannels of the microchip ELISA (or regions within the microchannels), for example by executing a search algorithm.

More specifically, the search algorithm executed by the application uses color intensity of the image pixel values as red, green and blue pixel values using a RGB color model, in which red, green, and blue pixel values vary from 0 to 255. As is known in the art, when red, green, and blue pixel values are at 255, the color signal is saturated. In the search algorithm, a threshold is defined for the red pixel values (“R values”) extracted from an area surrounding the microchannels in the image and used as a base value. The algorithm then selects regions within the imaged microchannels, defined by having R values that are lower than the threshold, for data analysis. This threshold may be based on previously obtained images and can be implemented as a user-defined modifiable parameter. In some instances, a default threshold value of 70 is used (based on previous observations). The algorithm determines whether a first region starting from a selected pixel is a continuous region with low R values. The application offsets the first region and continues to a second region and so on until a region is found that has continuous R values below the threshold. This region is then determined as an imaged microchannel region, or a detection region. The number of detection regions determined can correlate to the number of microchannels present on the microchip ELISA. In addition, in some implementations, blue pixel values, green pixel values, or other wavelengths can be used for detection region selection and for any other analysis steps discussed below in place of R values. Furthermore, the image data can be converted to relative score values for use in data analysis.

In some instances, imaged microchannel regions may have a low color intensity and do not illustrate clear difference from the background of the image. To facilitate region selection by the mobile application, some images maybe modified to add markers or indicators next to the imaged microchannels to assist the selection of the detection regions. In addition, markers can be physically placed on the microchips to facilitate detection region recognition during image analysis. In some instances, the mobile application assumes that the captured images are oriented horizontally with small rotation angles. As such, assumptions can be made that the detection regions are axis-aligned “red rectangles” and each region in the same image is vertically aligned.

To minimize the illumination difference between analyzed images, the color intensity can be normalized based on the difference in the backgrounds (specifically, regions excluding the detection regions) between one or more stored calibration images (“baseline images”) and the sample image. The application selects a typical background region from the sample image and compares the R values therein to an average of R values from the background regions of the calibration images. The R values from the selected region in sample image is then offset, or normalized, by deducing the R value difference. In some instances, microchips can include a separate calibration channel or microchannel and relative R values can be determined from the calibration microchannel and the ELISA microchannels for normalization.

The application then applies an average of the normalized R values from the detection region against a baseline curve relating R values to analyte concentrations. The baseline curve is calculated by determining a regression line correlating R values of the calibration images to known HE4 concentrations of the calibration images. Example calibration images include images of microchip ELISAs prepared with sample concentrations, such as 1,250, 625.0, 312.5, 156.3, 78.1, 39.1 and 19.5 nanograms per milliliter (ng/mL). For example, these calibration samples are previously imaged during a “baseline curve calibration mode” of the application, selected through a settings page of the application, to calculate the baseline curve and store background R values for normalization purposes (as described above). In some instances, the calibration images can be taken or loaded in order starting from higher concentrations to lower concentrations for regions to be assigned with the correct concentration values. In addition, the application can receive new calibration images to recalibrate the baseline curve at any time (for example, by calculating R values of the new images and updating baseline curve regression parameters).

Using the baseline curve against the normalized R values from the detection regions, the HE4 concentration of the imaged sample can be determined. With reference to process block 42, the HE4 concentration is then reported, for example by displaying the concentration on the mobile device screen. An example display 68 of reported HE4 concentration on a mobile device 22 is illustrated in FIG. 6. An image 70 displayed by the mobile device 22 of FIG. 6 illustrates a background region “1” and three detection regions (a top region “2”, a middle region “3”, and a bottom region “4) differentiated by the application. A results section 72 displayed by the mobile device 22 illustrates R values for each detection region and the background region, baseline curve regression line parameters used to correlate the R values to HE4 concentrations, and determined HE4 concentrations for each detection region.

The application may also compare the analyte concentration to a threshold concentration to determine if the analyte concentration is above the threshold concentration, indicating a positive (or preliminary positive) ovarian cancer result, or if the analyte concentration is below the threshold concentration, indicating a negative (or preliminary negative) ovarian cancer result. The application can then also display the positive/negative result on the mobile device screen. In addition, the application can receive demographic or epidemiologic variables (for example through message texting and data sharing via mobile networks or through direct user input into the mobile device) and use such variables to facilitate diagnosis. In one example, malignancy prediction can incorporate menopausal status as a variable.

As discussed above, process blocks 34-42 can be executed by an application stored on the mobile device or the additional device. For example, an image processing software application for use with the additional device can be created and/or executed using tools such as MATLAB or IMAGEJ. In another example, a mobile device application can be applicable to, or more specifically, can be capable of being downloaded to and executed by, various smart phone platforms, such as an Windows Phone 7 operating system. In addition, a smart phone operating system emulator (for example, based on Visual C#2010 Express, Microsoft Visual Studio®) downloaded on a computer or the additional device can be used to execute the mobile device application.

Integration of the mobile application with a mobile device, as discussed above, enables immediate processing of the microchip ELISA results, which eliminates the need for a conventional bulky, expensive spectrophotometer. As a result, the above microchip ELISA preparation and detection method to measure ovarian cancer biomarker concentrations in urine can potentially be operated by a healthcare worker with minimal training. This detection module has the potential to realize point-of-care (POC) testing in both developing and developed countries, and can be potentially used for early detection of ovarian cancer among high-risk populations as well as follow-up treatment monitoring at the POC or primary care. Identification of cancer patients among high-risk populations would potentially enable early treatment, and as a result, a reduction in mortality rate. Non-invasive urine testing also offers easy sample collection, enabling frequent testing (for example, as a pre-screening tool). In addition, the integrated mobile application can be employed in both resource-rich and resource-limited settings because of increasingly available mobile networks, whereby the appropriate clinical information can be instantly and remotely transferred between patients and physicians. This can also allow remote patient diagnosis and instructing. For example, a patient performs the procedure, sends sample images to a physician or caregiver, and receives instructions from the physician (manually, or from an automated program response based on the image analysis) to perform specific actions, such as to ingest a particular medication, to cease a particular medication, to see the physician immediately for follow-up, etc. Furthermore, the above method can be broadly applied as biotechnological tool for any disease having a reasonably well-described ELISA biomarker in biological samples such as urine or blood. For example, other ovarian cancer biomarkers or biomarkers indicative of other diseases can be detected using methods of the present invention.

A study was performed to demonstrate the feasibility of the above-described method of the present invention and compare HE4 concentration results obtained from the above-described method, using both mobile device camera-based imaging and CCD-based imaging, with microplate ELISA methods. The specific methods used and results from the study are described in the following paragraphs.

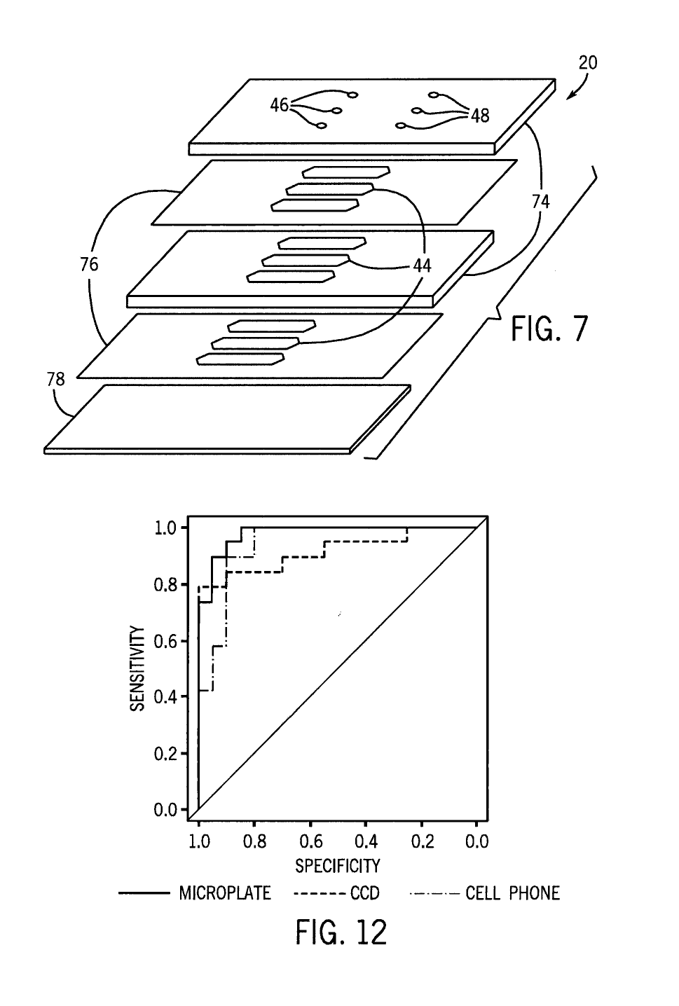

In the study, microchips were fabricated according to a non-lithographic technique. Specifically, as shown in FIG. 7, a fabricated microchip 20 included polymethyl-methacrylate (PMMA) 74 (McMaster Carr, Atlanta, Ga.) and double-sided adhesive film 76 (iTapstore, Scotch Plains, N.J.). The PMMA 74 and the film 76 were first cut using a laser cutter (VersaLaser™, Scottsdale Ariz.). The pieces 74, 76 were cut with dimensions of about 24 millimeters by about 40 millimeters. On the top of the PMMA base 74, an inlet 46 and outlet 48 were cut with a diameter of about 0.375 millimeters. Then, two layers of PMMA 74 were assembled onto a polystyrene Petri dish 78 (BD Biosciences, San Jose, Calif.) via two layers of double-sided adhesive film 76, forming three microchannels 44. These microchannels 44 had dimensions of about 2 millimeters wide by about 12 millimeters long by about 1.5 millimeters deep.

With respect to microchip ELISA preparation and test performance in the study, urinary peptides derived from human protein HE4 were modified for enhanced antigenicity. The optimized peptide sequences (CSLPNDKEGSCPQVNINFPQL) were synthesized and used to generate a rabbit polyclonal antibody (21st Century Biochemicals, Inc. Marlborough, Mass.) as the capture antibody. After application of about 100 microliters of test samples to each microchannel, the microchip was incubated at room temperature for an hour. Following the sample incubation, the microchip was blocked with 3% bovine serum albumin (BSA, m/v, Fischer Scientific, Pittsburgh, Pa.) at 37 degrees Celsius for an hour. An anti-HE4-rabbit primary antibody (0.61 milligrams/milliliter (mg/mL)) was diluted in 1:50,000 in 3% BSA blocking buffer and injected into the microchip for incubation at 37 degrees Celsius for an hour. The secondary antibody, anti-rabbit-HRP (1 mg/mL, Abeam, Cambridge, Mass.), was diluted in 1:3,000 in Tris-buffered saline and Tween-20 (0.05%), and incubated at 37 degrees Celsius for an hour. Following each incubation step, the microchip was washed three times by injecting 200 microliters of an ELISA washing buffer (50 mM Tris-HCl, 150 mM NaCl and 0.05% Tween-20). For color development, 100 microliters of one-Step ultra TMB (Thermo Fisher Scientific Inc., Waltham, Mass.) was injected, and incubated at room temperature in the dark for 9 minutes.

The above procedure was also followed for preparation and test performance of a conventional 96-well microplate ELISA. However, for the microplate ELISA, following addition of 100 microliters of TMB, the microplate was incubated for 15 minutes at room temperature, and the color development was stopped by adding 100 microliters of 1 M sulfuric acid (H2S04).

Both known-concentration test samples and clinical test samples were used in the study. The known-concentration test samples were prepared using pure HE4 peptide antigen serially two-fold diluted in sodium bicarbonate (0.1 M, pH 9.7) to provide final concentrations of 1,250, 625.0, 312.5, 156.3, 78.1, 39.1 and 19.5 ng/mL. The clinical test samples included forty de-identified and discarded clinical urine samples obtained from Brigham and Women's Hospital (Boston, Mass.). The clinical test samples were diluted 20 times before testing.

With respect to microchip ELISA imaging in the study, the optical color development on the microchip was imaged using a mobile device camera and a portable lensless CCD. Specifically, a cell phone (Sony Ericson i790) with a 3.2 megapixel camera, and a lensless CCD (IPX-llM5, Imperx, Boca Raton, Fla.) with a resolution of 11 million pixels were utilized.

With respect to microchip ELISA analysis in the study, the color intensity of red, green and blue pixel values was measured for each microchannel. This was accomplished for the cell phone results using an integrated cell phone application and for the CCD results using a connected laptop with a customized MATLAB (MathWorks, Natick, Mass.) code. For validation, the selected region from each microchannel by the cell phone application was also transferred to a laptop and processed using the customized MATLAB code. It was noted that the compressed jpeg format of the cell phone images that were transferred to the laptop caused negligible R value differences within less than 1%. The source code for the mobile application was written in C# for a Windows Phone 7 operating system. A sample of the MATLAB code is shown below:

for i=1:34

str=strcat(int2str(i),‘.jpeg’);

I=imread(str);

Ired=1;

Igreen=1;

Iblue=1;

Ired(:,: 2)=[ ];

Ired(:,: 2)=[ ];

Igreen(:,: 1)=[ ];

Igreen(:,: 2)=[ ];

Iblue(:,: 1)=[ ];

Iblue(:,: 1)+[ ];

r=mean2(Ired);

g=mean2(Igreen);

b=mean2(Iblue);

std=std2(Iblue);

rgb(i,1)=r;

rgb(i,2)=g;

rgb(i,3)=b;

rgb(i,4)=std;

end

rgb

xlwrite(‘rgb.xls’,rgb);

From both the cell phone application and the MATLAB code, the red, green, and blue pixel values of each channel were reported as a mean value plus/minus a standard deviation. The pixel values were correlated with known HE4 concentrations using the known-concentration test samples to calculate a baseline curve. The concentrations were log-transformed, since they were not in normal distribution, to create the baseline curve. The pixel values of the clinical test samples were applied to the baseline curve to determine their respective sample HE4 concentrations.

With respect to microplate ELISA analysis, conventional analysis was performed for calibration using the known-concentration samples and for determining the HE4 concentrations of the clinical test samples. The color intensity of the microplate ELISA results was measured by a microplate reader (BioTek, Winooski, Vt.) at a wavelength of 450 nanometers. In addition, the resultant color solution from each microchannel was transferred to a 96-well microplate, and the optical density (OD) was measured using a spectrophotometer.

The above-described analysis provided HE4 concentration results, from both the known-concentration samples and the clinical test samples, from microchip ELISA cell phone images through the cell phone application and MATLAB, from microchip ELISA CCD images through MATLAB, from microchip ELISA results transferred to a microplate through conventional optical density analysis, and from microplate ELISA through conventional optical density analysis. Data analysis in the study focused on red pixel values (R values), since they demonstrated the widest range of color intensity, as measured using the CCD and cell phone camera. The following paragraphs discuss results of the above-described study through comparison one or more of the above HE4 concentration results.

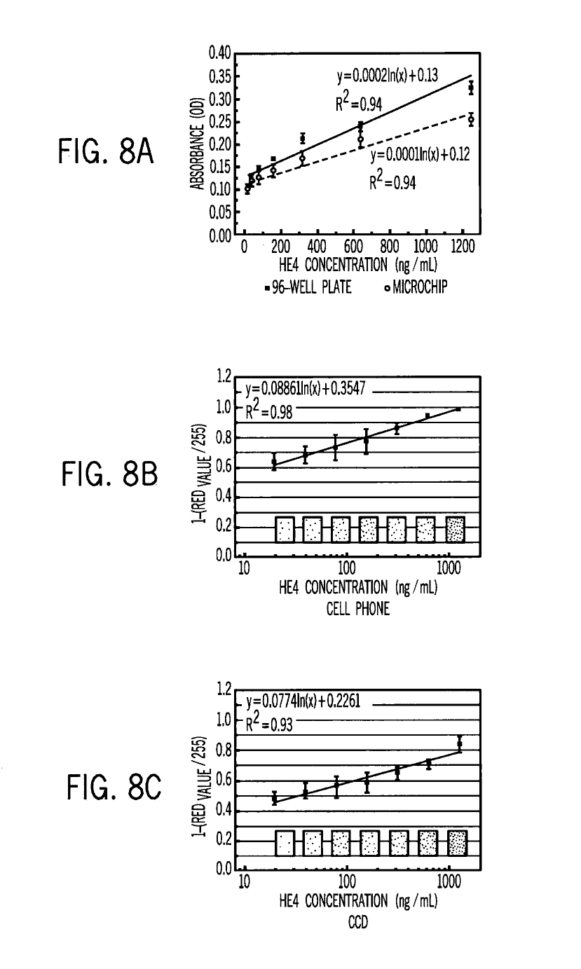

With respect to optical density measurements from the microchip ELISA solution transferred to a microplate and the conventional microplate ELISA, both results presented similar linearity for HE4 peptide concentrations of 1,250, 625.0, 312.5, 156.3, 78.1, 39.1 and 19.5 ng/mL, each with a coefficient of determination (R̂2) value of 0.94, as shown in FIG. 8A. These results indicated that ELISA was reliably carried out on the microchip with a performance comparable to that on a 96-well microplate. Because of increased surface-to-volume ratio in the microchip, 9 minute TMB incubation was chosen for the microchip ELISA throughout the study to avoid saturated signals. Lower OD readings were observed for the microchip ELISA than microplate ELISA OD readings, which was most likely due to the shorter incubation of TMB on-chip compared to 15-minute incubation on-plate.

With respect to the cell phone application and MATLAB analysis code, both systems relied on the analysis of red, green, and blue pixel values of the color solution developed on-chip as a result of the microchip ELISA reaction. In the study, the red pixel value (R value) had the widest changes among the tested standard concentrations ranging from 1,250 to 19.5 ng/mL, and the changes in red pixel values were strongly correlated with the HE4 concentration. The following table illustrates average R values obtained from the known-concentration samples through the mobile application and MATLAB.

TABLE 1
HE4 PEPTIDE STANDARDS TESTED BY CELL-PHONE BASED
MICROCHIP ELISA USING MOBILE APPLICATION AND MATLAB

| HE4 Concentration | Average R values by | Average R values by |
|---|---|---|
| (ng/mL | MATLAB | mobile application |
| 1250.0 | 2.662 | 2.165 |
| 625.0 | 14.737 | 14.708 |
| 312.5 | 35.597 | 35.610 |
| 156.3 | 58.823 | 57.865 |
| 78.1 | 69.038 | 68.213 |
| 39.1 | 82.520 | 81.805 |
| 19.5 | 93.251 | 92.723 |
| 0 | 121.014 | 121.090 |

In correlating the average R values shown above in Table 1 with the known HE4 concentrations, the integrated mobile application reported an R̂2 value of 0.98 for the baseline curve over a range of 19.5-1,250 ng/mL, as shown in FIG. 8B. In the CCD based approach, the MATLAB code provided an R̂2 value of 0.93 for the baseline curve, as shown in FIG. 8C, which was comparable to that obtained in microplate ELISA analysis (as shown in FIG. 8A).

In three additional independent experiments, the linearity of the baseline curve from the cell phone application was highly comparable with R̂2 values of 0.938, 0.992, and 0.972, respectively. These results indicated that the cell-phone based microchip ELISA method was reproducible despite multiple testing steps involved in the prototype. More specifically, during the clinical testing, experiments were carried out by two operators performing each ELISA step through manual pipetting of reagents. There was no observed significant difference in the concentration of HE4 obtained by the two operators. At the POC, the reagent flow steps may be automated with the aid of a micropump (as described above), therefore minimizing the added complexity of pipetting to the method.

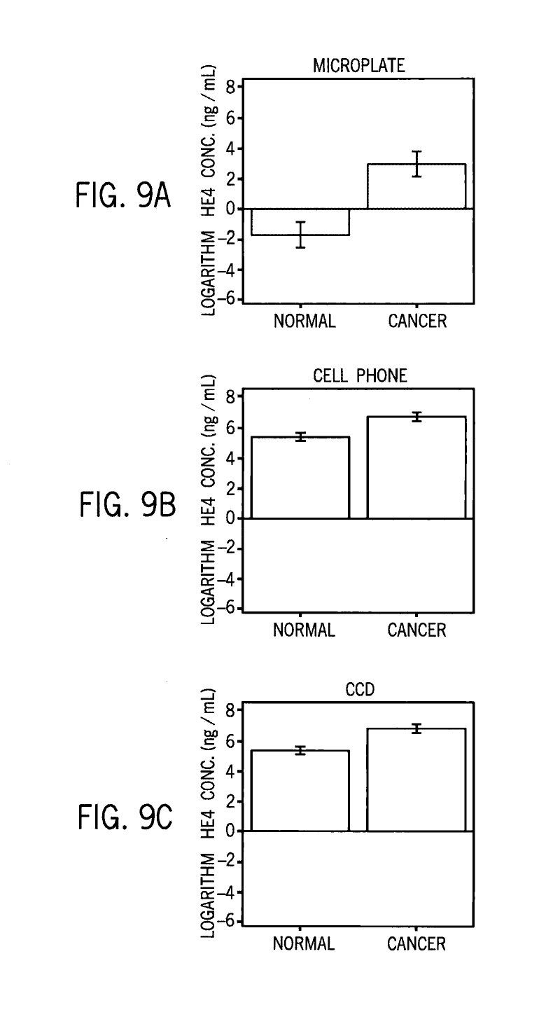

With respect to clinical testing validation, to determine whether the two groups of clinical test samples—ovarian cancer patients (prior to surgery, n=19) and age-matched healthy controls (n=20)—were within the same distribution, a two-sample Wilcoxon ranks-sum test was used. It is noted that one clinical test sample from an ovarian cancer patient was excluded for statistical analysis because of its aberrant urine creatinine concentration. For the microplate method, the means, standard errors of the sample mean (SEMs), and 95% CIs were −1.69, 0.31, [−2.29,−1.08] for normal urine samples and were 2.95, 0.27, [2.42, 3.47] for cancer urine samples, as shown in FIG. 9A. For the cell phone method, the means, SEMs, 95% CIs were 5.35, 0.09, [5.17, 5.52] for normal urine samples and were 6.68, 0.09, [6.50, 6.86] for cancer urine samples, as shown in FIG. 9B. For the CCD method, the means, SEMs and 95% CIs were 5.44, 0.08, [5.27, 5.61] for normal urine samples and were 6.79, 0.13, [6.54, 7.03] for cancer urine samples, as shown in FIG. 9C. In the results, since HE4 concentrations were log-transformed for analysis, concentrations below 1 ng/mL appear as negative values. P-values of two-sample Wilcoxon tests for these three methods were all <0.001. The low p-values obtained by microchip ELISA and microplate ELISA indicated the logarithm-transformed HE4 concentrations for the majority of cancer urine samples were significantly greater than that of normal urine samples.

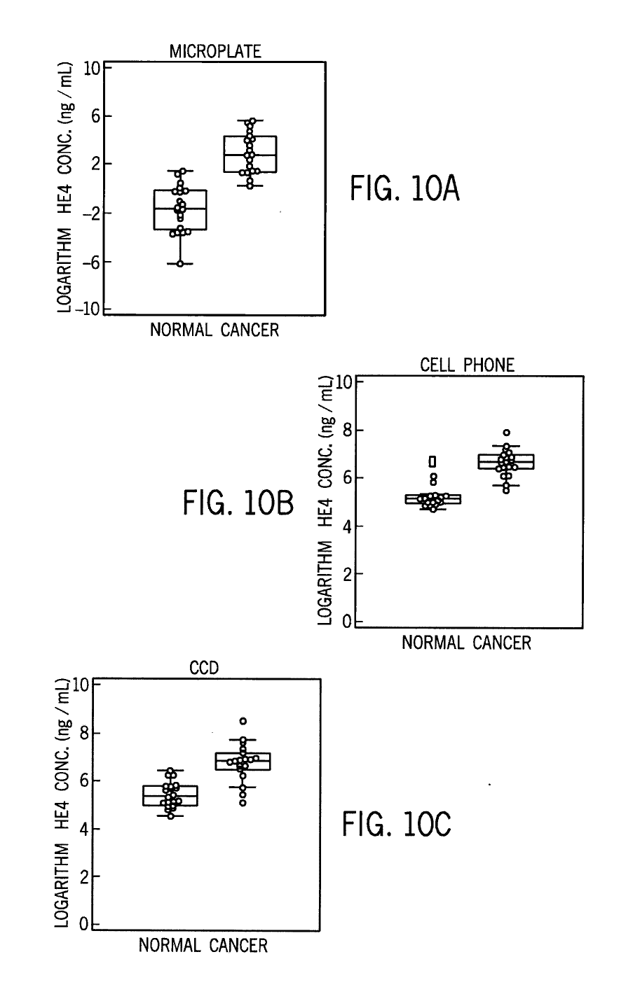

Box-Whisker analyses also showed that the detected level of HE4 concentrations, after log-transformation, in the 39 clinical urine samples was significantly (p<0.001) elevated in the ovarian cancer group compared to the control group using the cell phone-based and CCD-based microchip ELISA, and the conventional microplate ELISA, as shown in the parallel box-plots of FIGS. 10A-10C. The minimum, first quartile, median, third quartile, and maximum of the logarithm-transformed HE4 concentrations were −6.215, −1.623, 0.631, 0.572, 2.729, and 5.624, respectively, for the microplate ELISA method, as shown in FIG. 10A, 4.755, 5.188, 6.101, 5.999, 6.751, 7.965, respectively, for the cell phone-based microchip ELISA method, as shown in FIG. 10B, and 4.550, 5.254, 5.844, 6.095, 6.865, 8.511, respectively, for the CCD-based microchip ELISA method, as shown in FIG. 10C. A few outliers were observed for the logarithm-transformed HE4 concentrations measured by cell phone-based and CCD-based microchip ELISA methods. Box-Whisker analyses were performed using MedCalc Version 11.5.1 (Mariakerke, Belgium).

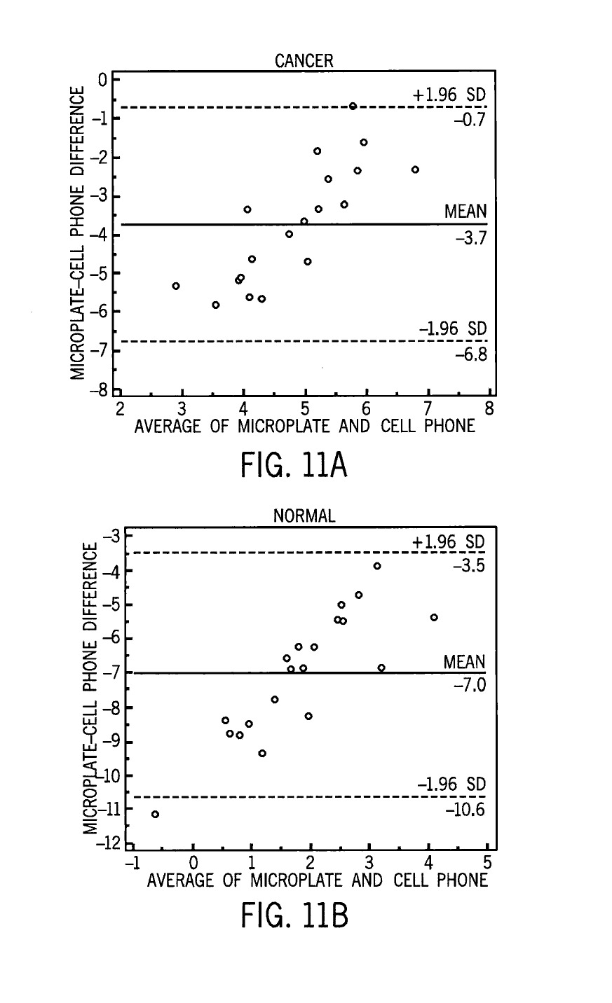

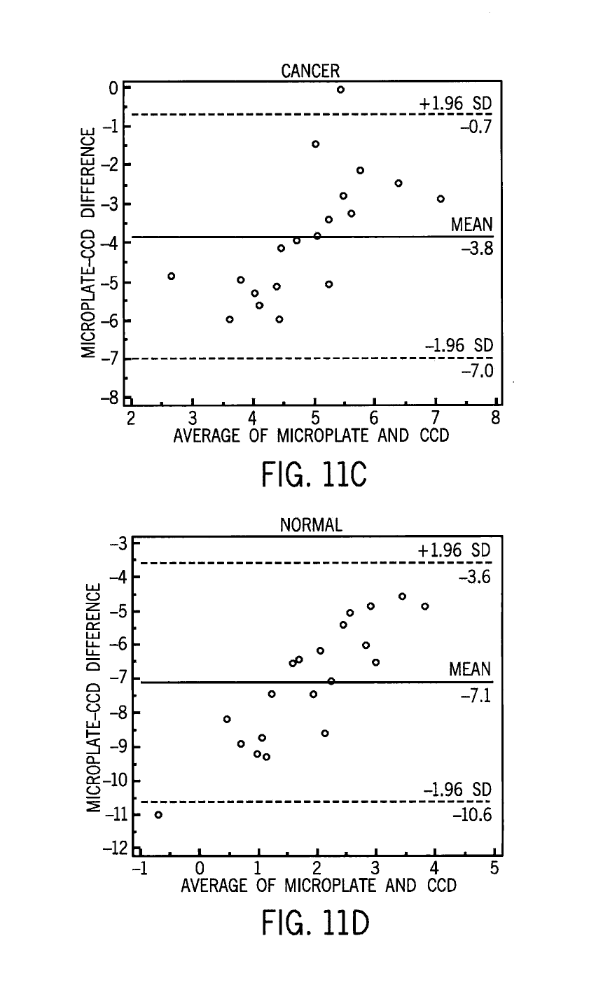

The microchip ELISA and conventional 96-well microplate ELISA HE4 concentration results for both cancer and control groups were also compared using the Bland-Altman analysis method. The results, as shown in FIGS. 11A-11D indicated that cell phone-imaged microchip ELISA had bias in measuring log-transformed HE4 concentrations compared to microplate ELISA in both cancer patients (−0.7 to −6.8) and normal healthy controls (−3.5 to −10.6). Microchip CCD ELISA had bias in measuring log-transformed HE4 concentrations compared to microplate ELISA in both cancer patients (−0.7 to −7.0) and normal healthy controls (−3.6 to −10.6). Log-transformed HE4 concentrations measured by microchip cell phone ELISA and microchip CCD ELISA were in agreement, with a bias of 0.68 to 0.89 in cancer patients and a bias of 0.66 to 0.84 in normal healthy controls.

The observed bias in HE4 quantification using microchip ELISA (both CCD and cell phone) compared to microplate ELISA indicates that there are differences in quantifying urinary HE4 between these two methods. This is most likely due to batch-processing of the clinical samples on microchips. Unlike the 96-well microplate, color development was not stopped on the microchip since the stop solution would have removed the color solution. Since the time window to take images before saturated signals occurred was narrow, the 48 samples (clinical and standard samples, tested in duplicates) were divided into 6 batches with 8 samples per batch. Despite this, slightly over-developed signals for some cases were observed. The slightly over-developed signals may have contributed to higher quantification of these samples on-chip than on the microplate. In comparison, the HE4 measurements by microchip ELISA methods were in agreement. Considering the variation between batch processing, fully automated microchip ELISA may be beneficial to reduce variation and improve the correlation between microchip ELISA and microplate ELISA.

To further evaluate the prediction power of the urine HE4 concentration, receiver operating characteristic (ROC) curves were constructed, as shown in FIG. 12, and calculation of the area under the ROC curves (AUROC), sensitivity given specificity, and their 95% confidence intervals (CIs) was performed. The 95% CIs of these parameters were based on 10,000 bootstrapping samples. In each bootstrapping replicate, 39 subjects were first randomly sampled with replacement from the original data set. The AUROC and sensitivity given specificity was then calculated based on this replicate. The 2.5 and 97.5 percentiles of the 10,000 estimated AUROC (sensitivity given specificity) were used as the lower and upper limits of the 95% CI. Statistical Software R (available at http://www.r-project.org/) was used to estimate the sensitivity given specificity and their 95% bootstrapping confidence intervals. A sensitivity of 94.7%, 89.5% and 84.2% for the conventional 96-well microplate ELISA, microchip cell phone ELISA and microchip CCD ELISA, respectively, was a observed with the specificity set to 90% for all three approaches. The AUROCs were 0.979, 0.940 and 0.916 for microplate, cell phone microchip ELISA and CCD microchip ELISA, respectively. Since the AUROCs were close to the maximal possible value of 1, the urine HE4 concentration measured by all three methods had high accuracy to identify ovarian cancer patients from normal controls. Therefore, despite the bias in HE4 quantification between the microchip ELISA methods and microplate ELISA methods, the microchip ELISA methods were still able to differentiate ovarian cancer patients from normal controls.

The observed sensitivity was comparable to that obtained in a previous study using conventional microplate ELISA, which concluded a sensitivity of 86.6% for early stage (I/II) and 89% for late stage (III/IV), when the specificity was set to 94.4%. In this previous study, a combination HE4 biomarker (weighted average of urinary HE4 level and HE4/creatinine ratio) was used for calibration in the previous study. Currently, there is no standard method to calibrate urine biomarkers. However, it has been reported that the urinary creatinine level may not be the ideal calibrator for urine biomarker normalization, especially for cancer patients at advanced stages, who may have renal failure or impairment due to cancer progression or chemotherapy intervention. Since the clinical test urine samples were collected from late stages (III and IV) of ovarian cancer patients, no creatinine-based calibration was performed in the study described above.

The above results demonstrate the feasibility of using a mobile device or CCD to facilitate microchip ELISA-based non-invasive detection of HE4 concentrations in urine. The use of a mobile device or CCD for microchip ELISA readout and a mobile application that measures the color intensity and reports the analyte concentration on the mobile device screen allows for on-site measurement and analysis of ELISA results without expensive, specialized instruments (e.g., a microplate reader connected to a computer). The microchip ELISA, either coupled with mobile device detection or CCD detection, demonstrates the reliability to differentiate cancer patients from their healthy controls, as indicated by p values (<0.001) of the above-described study, therefore offering an inexpensive, reliable solution for POC ovarian cancer screening.

As discussed above, the methods of the present invention can be broadly applied as biotechnological tool for any disease or pathological condition having a reasonably well-described biomarker or analyte detectable through ELISA in biological samples such as urine, plasma, whole blood, serum, or saliva. Some examples include microchip ELISA-based p24 antigen detection (for example, from plasma) or CD4 cell count detection for detecting HIV, microchip ELISA-based KIM-1 detection or NGAL (neutrophil gelatinase-associated lipocalin) detection (for example, from urine) for detecting kidney injury, or microchip ELISA-based BDNF (brain-derived neurotrophic factor) detection for detecting traumatic brain injury. The above methods can also be applied to microchip ELISA-based E. coli detection, for example from whole blood samples. In some cases, multiple analytes can be detected on the same microchip ELISA. For example, urine HE4 and serum CA125 can both be tested simultaneously on a single chip to assist in cancer detection. It is noted that, for purposes of this disclosure, the term biomarker may encompass proteins, cells, pathogens, etc.

With reference to the microchip design discussed above, precise control of fluid flow is often required to ensure the success of the biological reaction (specifically, the ELISA). As a result, complicated flow procedures and/or sophisticated gating strategies are usually needed to deliver reaction components in a temporal and spatial manner to perform the ELISA. Although use of a micropump can alleviate most of these issues, current microchip designs still suffer from obstruction of air bubbles in microchannels, thereby inhibiting flow, and even so, in some recourse-limited locations, micropumps may be an impractical solution. Imprecise control of flow in such microfluidic devices may cause failure of biological reactions or inaccurate diagnosis under unfavorable flow conditions.

An aspect of the present invention is a chamber-based microchip design, considered a “micro-a-fluidic” approach, that can be easily adapted for accurate POC testing. This micro-a-fluidic approach does not involve precise fluid flow and thus significantly facilitates automation of complicated biological reactions (such as ELISA, or alternatively, polymerase chain reaction (PCR) testing). The micro-a-fluidic ELISA of the present invention elicits a substantially high sensitivity (less than 10 picograms/milliliter, pg/ml), which is about two- to four-fold higher than the sensitivity of conventional microplate ELISA. In addition, assay time using the micro-a-fluidic approach can be reduced to about 10 minutes, in comparison to 4-6 hours for conventional ELISA. It is noted that this assay time can be varied greatly and even further reduced, for example in the range of two to three minutes or down to ten seconds, based on reagent capabilities and other factors. Furthermore, the micro-a-fluidic ELISA can potentially be fully automated to realize “plug and play” type testing for POC diagnosis. Detection of micro-a-fluidic ELISA results can be achieved through conventional techniques or through mobile device or CCD-based imaging (for example, in accordance with methods of the invention described above). This can further increase the applicability of micro-a-fluidic based POC testing in resource-limited settings (specifically, by removing the need for a micropump as well as complicated imaging equipment).

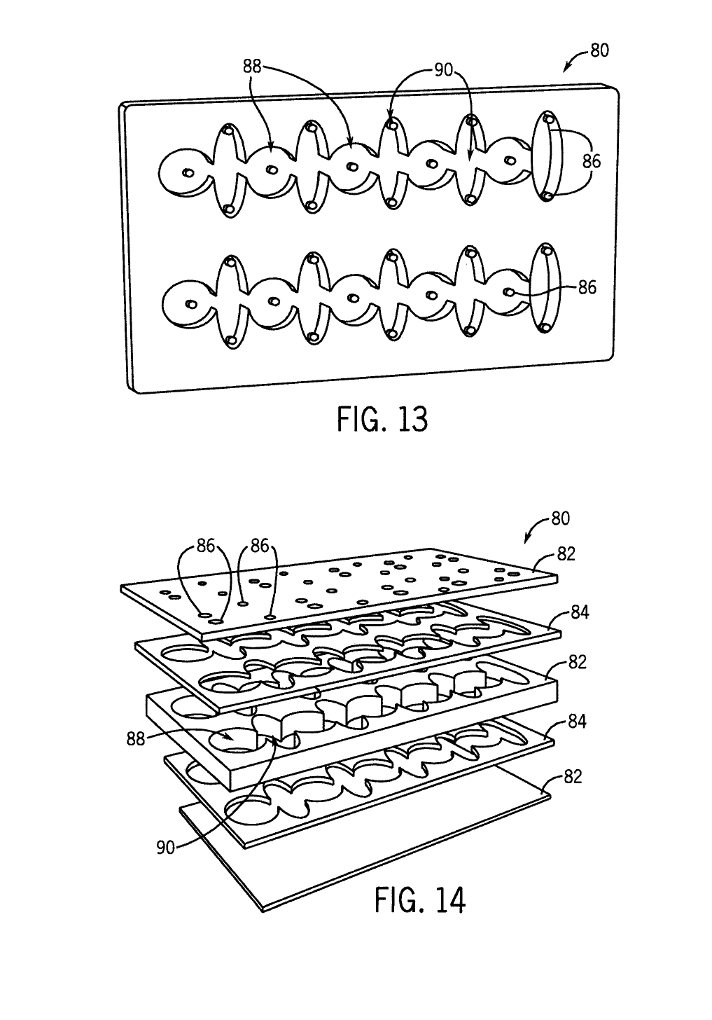

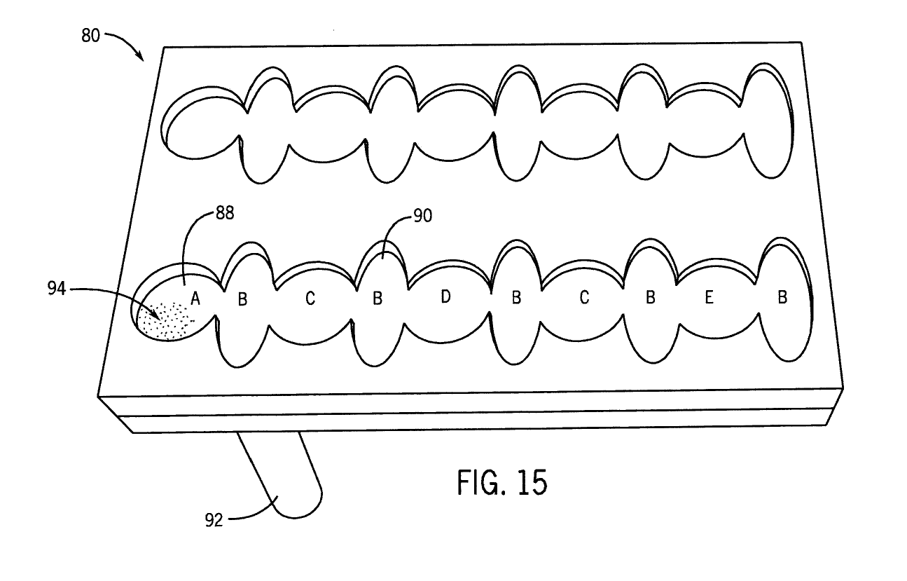

FIG. 13 shows a micro-a-fluidic ELISA 80, according to one aspect of the invention, for POC testing. The micro-a-fluidic ELISA 80 can be fabricated using a non-lithograph technique, and can be assembled using three layers of PMMA 82 and two layers of double sided adhesive (DSA) 84, as shown in FIG. 14. The top layer of PMMA 82 can include multiple circular openings 86 for sample and reagent loading. The middle layer of PMMA 82 and the two layers of DSA 84 can be cut to provide a plurality of chambers 88, 90. More specifically, as shown in FIGS. 13 and 15, the micro-a-fluidic ELISA 80 can include five circular chambers 88 (denoted chambers A, C, D, and E in FIG. 15) and five elliptical or rectangular chambers 90 (denoted chamber B). The elliptical chambers 90 can be loaded with glycerol oil to separate reagents for ELISA, which are separately loaded in the circular chambers 88.

In one specific example, the micro-a-fluidic ELISA dimensions are 42 mm in length, by 63 mm in width, by 6 mm in depth. The two outer layers of PMMA 82 are 1.5 mm thick, while the inner layer of PMMA 82 is 3 mm thick, and the two DSA layers 84 are 50 micrometers thick, providing chamber depths of 3 mm. The first circular chamber 88 (chamber A) has a radius of 4.5 mm, the other circular chambers 88 (chambers C, D, and E) each have radii of 3.5 mm, and the elliptical chambers 90 each include major and minor axes of 13.5 mm and 3.8 mm, respectively. The last elliptical chamber 90 (after chamber E) can have major and minor axes of 13.5 mm and 6.5 mm, respectively. The circular openings 86 have radii of 0.4 mm.

Referring to FIG. 15, in an example ELISA operation, a stack of magnets 92, (in some cases, having the same diameter as the circular chambers 88), are used to move magnetic beads or particles 94 conjugated with antigen-specific capture antibody across the chambers 88, 90. Chamber A can be first loaded with the magnetic beads 94 and an antigen of interest (totaling, for example, 100 microliters in volume). Each chamber C can be loaded with 100 microliters of a wash buffer, chamber D can be loaded with 100 microliters of detection antibody conjugated with horseradish peroxidase (HRP), and chamber E can be loaded with 100 microliters of TMB. The magnetic beads 94, including the captured antigen, can be moved from chamber A by the magnets 92 from one aqueous phase to another, each time crossing an oil barrier (chamber B), with a time frame of 1 minute in chambers C and D. In chamber E, the magnetic beads 94 are left for 5 minutes to facilitate color development. After the five-minute time period, the magnetic beads 94 are moved to the last rectangular chamber (containing glycerol oil) to avoid interference during imaging for colorimetric detection by the mobile device (or CCD, or digital camera). Thus, each step of the ELISA can be performed in separate chambers 88, which are separated from each other by an oil barrier. Comparative studies were performed using the above micro-a-fluidic ELISA techniques and conventional microplate ELISA, as further described below. The results of these studies demonstrate that micro-a-fluidic ELISA can be readily adapted for substantially any disease provided a validated biomarker.

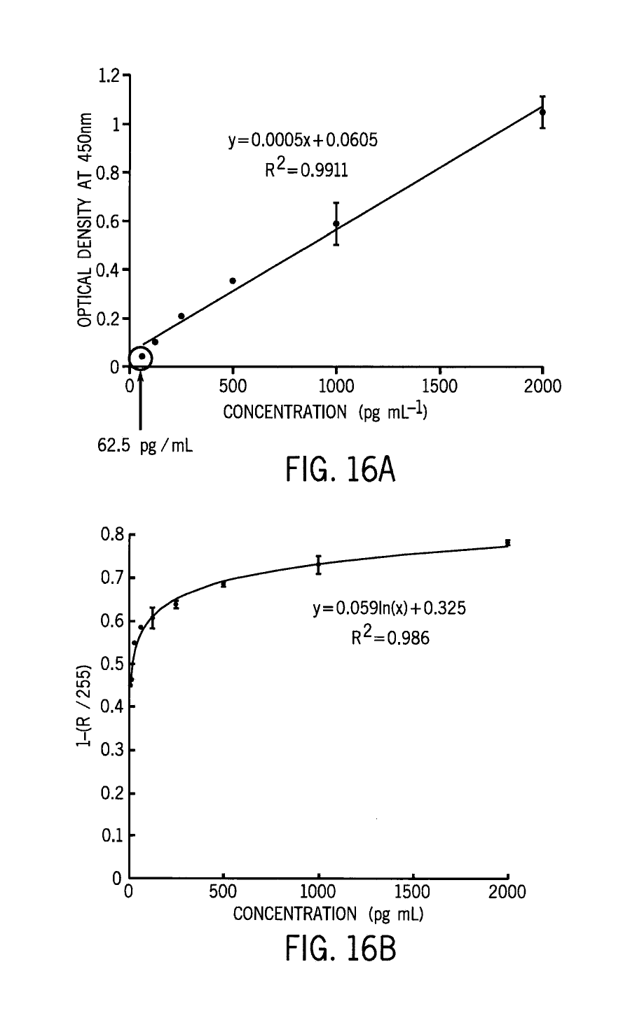

In one comparative study, BDNF concentrations ranging from 0 picograms/milliliter (pg/mL) to 2000 pg/mL were tested using conventional microplate ELISA and micro-a-fluidic ELISA. The micro-a-fluidic ELISA procedure in this case was completed in ten minutes. FIGS. 16A and 16B illustrate standard curves correlating the known BDNF concentrations to optical density measurements obtained for microplate ELISA via a spectrometer and to color intensity measurements obtained for micro-a-fluidic ELISA via mobile device-based imaging, respectively. Sensitivity for the conventional microplate ELISA was 62.5 pg/mL (as shown in FIG. 16A), while sensitivity for the micro-a-fluidic ELISA and mobile device-based imaging was 7.8 pg/mL (as shown in FIG. 16B).

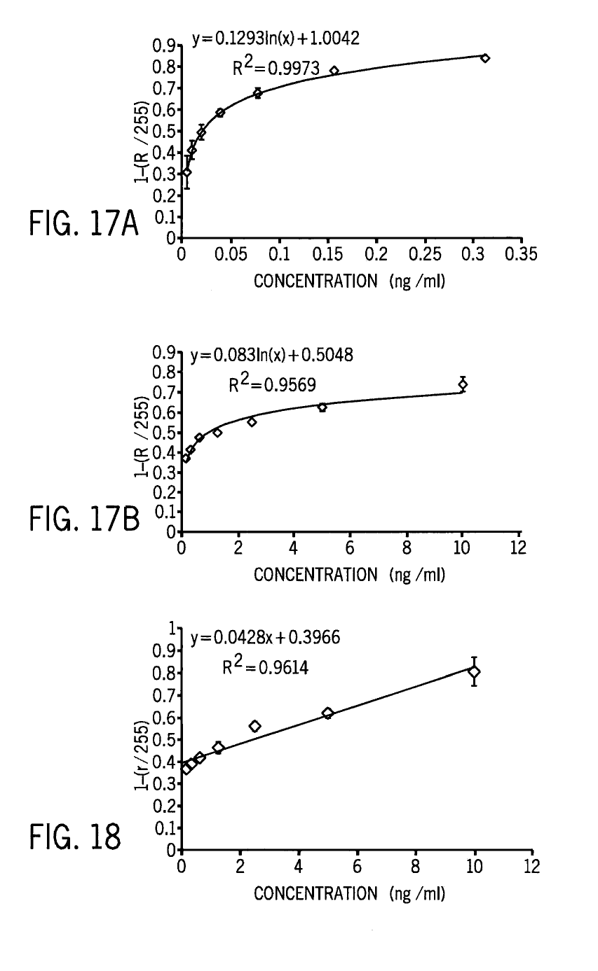

In another comparative study, KIM-1 concentrations were tested via micro-a-fluidic ELISA using concentrations ranging from 0 nanograms/milliliter (ng/mL) to 0.3125 ng/mL during a thirty-minute procedure and concentrations ranging from 0 ng/mL to 10 ng/mL during a ten-minute procedure. FIGS. 17A and 17B illustrate standard curves correlating the known KIM-1 concentrations to color intensity measurements obtained for micro-a-fluidic ELISA via mobile device-based imaging for the thirty-minute procedure and the ten-minute procedure, respectively. Sensitivity for the thirty-minute procedure reached 4 picograms/mL. During the ten-minute procedure, the detection limit was enhanced down to 156 pg/mL by reduced procedure time.

In another study, NGAL concentrations were tested via micro-a-fluidic ELISA using concentrations ranging from 0 ng/mL to 10 ng/mL, during a ten-minute procedure with a capture antibody concentration of 1.5 micrograms/mL. FIG. 18 illustrates a standard curve correlating the known NGAL concentrations to color intensity measurements obtained for micro-a-fluidic ELISA via mobile device-based imaging.

In yet another study, CD4 cell count was tested via micro-a-fluidic ELISA using an anti-CD4 antibody. In this study, protein G (PG) coated magnetic beads (approximately 1 micrometer in diameter) were used. For CD4 cell capture, mouse monoclonal anti-CD4 antibody was conjugated to the PG beads. Also, HRP conjugated secondary anti-CD3 antibodies were used. The study also relied upon visualization of captured CD4 cells on the magnetic bead surfaces, which was carried out through bright field imaging for validation.

During micro-a-fluidic ELISA testing, with reference to the micro-a-fluidic ELISA described above, a solution of phosphate buffered saline Tween-20 (PBST) with washing buffer was injected into each chamber C (“wash chambers”), anti-CD3 rabbit polyclonal secondary antibody conjugated with HRP was injected into chamber D (“secondary antibody chamber”), and TMB was injected into chamber E (“TMB chamber”). Surface tension from the interaction of the liquids with the PMMA and relatively strong intermolecular forces allowed the liquids to stay within each chamber. After these reagents were injected, mineral oil was injected into each elliptical chamber B, except for the first elliptical chamber B (between chambers A and C). Next, magnetic beads conjucated with mouse monoclonal anti-CD4 capture antibody and a blood sample was loaded into chamber A (“sample chamber”). PBST was added to fill the remaining volume of chamber A, and then mineral oil was added to the remaining empty chamber B.

Following this, magnets were applied under chamber A and then moved across the micro-a-fluidic ELISA. Specifically, upon the application of the magnets underneath chamber A, the magnetic beads, with captured CD4 T lymphocytes via antibody-antigen interaction, aggregated. The magnetic beads were actuated by the magnet to chamber B, crossing the elliptical chamber containing mineral oil. Mixing was performed by moving the magnet, and thus the magnetic beads, from end to end within the chamber. Moreover, the back and forth motion of the magnet attracts any residual magnetic beads left in the previous elliptical oil chamber B. After mixing for 1 minute in the wash chamber C, the beads were further actuated to chamber D containing the HRP-conjugated secondary antibody. While mixing for 1 minute in chamber D, the captured CD4 T lymphocytes interacted with the secondary antibody, forming an antibody sandwich structure. The magnetic beads were then moved to the second wash chamber C through another mineral oil elliptical chamber B via the magnetic. Following incubation, the magnetic beads were moved to chamber E containing TMB and accordingly mixed for 6 minutes. Due to the presence of HRP-antibody captured on the magnetic beads, the substrate TMB was digested and a blue color was developed. The magnetic beads were removed from chamber E into the last, larger elliptical oil chamber B. The magnetic beads were actuated back and forth for 1 minute to attract any residual beads left in chamber E. The total assay time was 10 minutes.

The micro-a-fluidic chip was immediately removed and put onto an LED-illuminated translucent white, acrylic plexiglass background inside a black plastic box, thereby reducing variations due to lighting. The micro-a-fluidic chip was imaged through a hole on top of the black box with a built-in camera in a cell phone, cropped using a software application, and the cropped images were analyzed using MATLAB as previously described. The black box with a LED and translucent white background provided a relatively isolated environment which reduced noise caused by differentiation of external lighting. Also, as the micro-a-fluidic chip is transparent, white backgrounds of the chip were cropped. These cropped white backgrounds were directly adjacent to the area of the blue cropped images from chamber E. The red pixel number (1−(Red Value)/255) of the white backgrounds was subtracted from the red pixel number of the blue cropped images, thereby normalizing the color values.

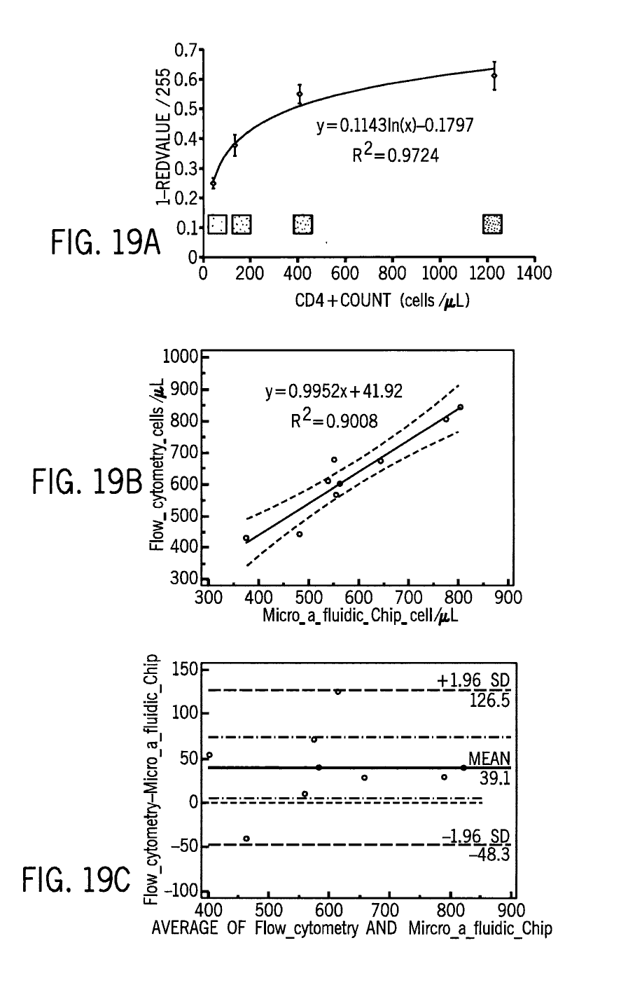

Samples (performed in triplicates) included both patient samples and calibration samples of known CD4 counts (obtained through flow cytometry). A logarithmic fit equation of the standard curve calculated using the calibration samples was used to correlate normalized pixel numbers of the patient samples and determine CD4 cell counts. FIG. 19A illustrates a standard curve correlating the known CD4 cell counts to color intensity measurements obtained for micro-a-fluidic ELISA via mobile device-based imaging. FIG. 19B illustrates a graph comparing CD4 counts obtained from micro-a-fluidic ELISA and flow cytometry. FIG. 19C illustrates a Bland-Altman analysis of the clinical results. In one portion of this testing, the limit of CD4 cell count detection using micro-a-fluidic ELISA was 88 cells/microliter (μL). Currently, the WHO standards for initiation of antiretroviral therapy (ART) is 350 cells/A. In light of this, CD4 micro-a-fluidic ELISA has the potential to be implemented in developing countries for ART monitoring.

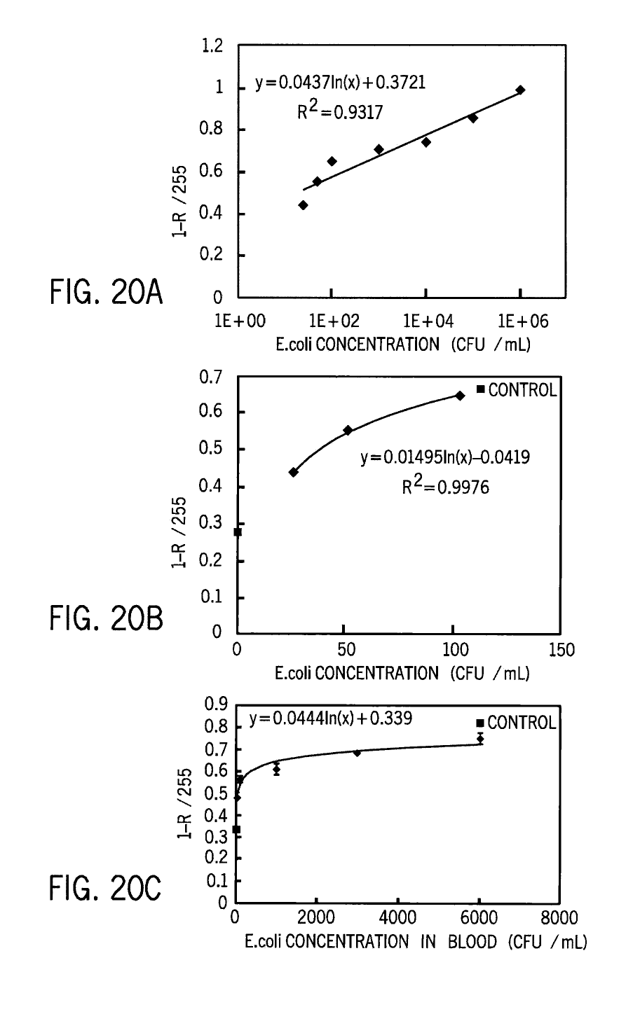

In another study, E. coli detection was tested via micro-a-fluidic ELISA using LBP (lipoplysaccharide binding protein) and an anti-LBP antibody. FIGS. 20A and 20B illustrate standard curves correlating known E. coli concentrations to color intensity measurements obtained for micro-a-fluidic ELISA via mobile device-based imaging. High correlation values, as shown in FIGS. 20A and 20B, illustrate strong sensitivity of E. coli concentrations by micro-a-fluidic ELISA testing. The standard curve of FIG. 20B shows a detection limit of 26 colony forming units (CFU)/mL. Furthermore, blood samples with varying concentrations of E. coli were tested using micro-a-fluidic ELISA. FIG. 20C illustrates a standard curve correlating the known E. coli concentrations in the blood samples to color intensity measurements obtained for micro-a-fluidic ELISA via mobile device-based imaging. As shown in FIG. 20C, a strong correlation was observed between the normalization of the red pixel color intensity and the E. coli concentrations. In addition, as shown in FIG. 20C, the sensitivity of E. coli concentrations, indicated by the limit of detection, was 25 CFU/mL in this case.

The above microchip designs and detection methods can also be applied to microchip-based neutrophil detection, for example to aid in the detection of peritonitis in peritoneal dialysis (PD) patients. In particular, end stage renal disease (ESRD), which affects approximately 8,000 patients per million worldwide, usually requires kidney replacement or dialysis to preserve any residual renal function. In comparison to traditional hemodialysis, peritoneal dialysis affords higher patient satisfaction in terms of cost, mobility, and convenience for medical treatment. However, one of the major risk factors of peritoneal dialysis is the occurrence of peritonitis (inflammation of the peritoneum) as a result of an infection. Currently, diagnosis of peritonitis is difficult to achieve until the final stages of infection. With a mortality rate of 6% among those suffering from peritonitis, patients are forced to switch to hemodialysis. Accordingly, in order to avoid peritonitis and its complications, the ability to predict when patients are in danger of developing peritonitis can be helpful for a physician to determine when to commence treatment.

Peritonitis is clinically defined as the occurrence of a turbid effluent in the dialysate containing more than 100 white blood cells (WBC)/μL, of which more than 50% are polymorphonuclear cells (neutrophils). Furthermore, detection of a substantial increase in the number of neutrophils in peritoneal fluid can be used as an indication of the degree of infection. Thus, one aspect of the invention includes a method for specifically and efficiently capturing neutrophils on a PD microchip from a PD patient sample, imaging the PD microchip, analyzing the image to determine a neutrophil concentration, correlating the neutrophil concentration to the presence of peritonitis or a degree of infection, and/or reporting this determination (for example, peritonitis present, peritonitis absent, high degree of infection, low degree of infection, etc.).

A study was performed to demonstrate the feasibility of the above-described method of the present invention and compare neutrophil concentration results obtained from the above-described method using CCD-based imaging with conventional fluorescence-activated cell sorting (FACS) methods. The specific methods used and results from the study are described in the following paragraphs.

In the study, microchips were fabricated according to a non-lithographic technique. Specifically, a fabricated microchip included laser-cut PMMA layers (3.175 millimeters in thickness) and double-sided adhesive film layers (80 micrometers in thickness). For each microchip, microchannels were prepared by first injecting 100 microliters (μL) of silanization solution followed by a 30-minute wait period at room temperature. The microchip was then washed with 100 μL of 100% ethanol, followed by injection of 100 μL of GMBS solution and then a 35-minute wait period at room temperature. After the wait period, the microchip was washed with 100 milliliters of ethanol and then 100 μl of phosphate buffered saline (PBS), followed by injection of 10 of neutravidin solution and then a 1-hour (or overnight) wait period at 4 degrees Celsius. After the wait period, the microchip was washed with 100 μL PBS and 100 μL 1% BSA-PBS, followed by a 1-hour wait period at 4 degrees Celsius. After the wait period, the microchip was washed with 100 μL PBS and the microchannels were injected with 15 μL of diluted CEACAM antibody (anti-CD66b), which allows for selective binding to neutophils, followed by a 30-minute wait period at room temperature, and then another PBS wash. The PBS washes, 30-minute wait period, and CEACAM antibody injection were repeated one more time, and then the microchip was soaked in PBS in a Petri dish wrapped in parafilm for storage until testing.

Samples in the study were prepared by spiking PD fluid with known neutrophil concentrations (using a stock WBC solution obtained from whole blood). The samples ranged in neutrophil concentrations from 25-1000 neutrophils/μL. The procedure for sample injection included running 100 μL PBS through microchannels manually, then injecting 10-100 μL of the PD sample at 2 μL/minute with a syringe pump, incubating the microchip for 10 minutes, and running another 100 μL of PBS at 5 μL/minute. For FACS analysis, a solution of 1 μL of CD66B plus 49 μL of DAPI was also run through the microchannels at 5 μL/minute, followed by a 30-minute incubation period, and another 100-μL PBS run at 5 μL/minute. After this procedure, the microchips were imaged using a CCD and a fluorescent microscope.

CCD images were analyzed using an application for interpreting the color image to count cells on the microchip. Specifically, a band-pass filter was applied to the CCD image data and the filtered data was then converted to a binary image to emphasize the cells, in particular, to create “halos” throughout the filtered image where cells were located. The number of cells was determined by detecting and counting the halo shapes present in the filtered image. The fluorescent microscope images were analyzed using FACS through bright field image analysis, GFP analysis for CD66b detection (which is specific to neutrophils), and CytoS analysis for DAPI detection (which is specific to all types of cells).

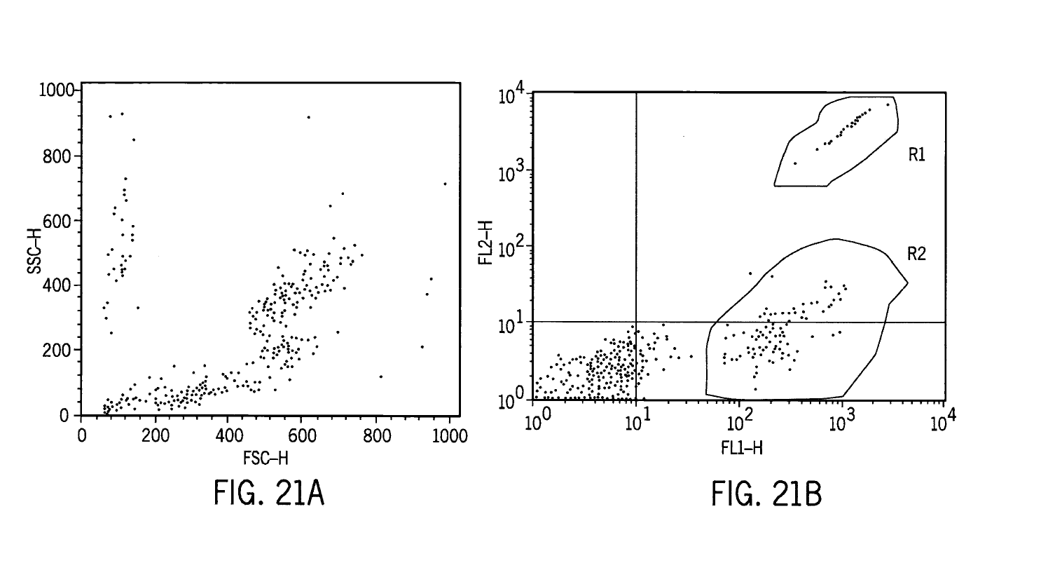

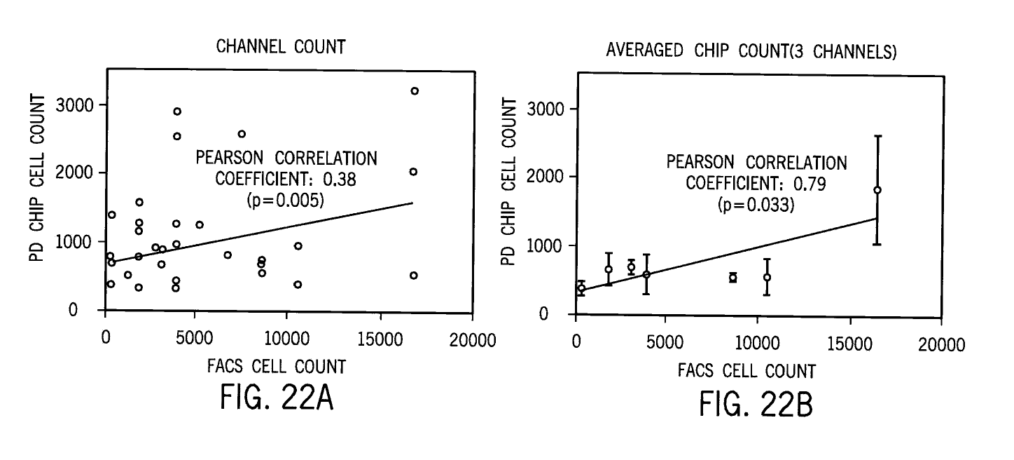

FIGS. 21A and 21B illustrate FACS results from a known-concentration sample prepared using the above procedure. The FSC-H vs. SSC-H plot of FIG. 21A illustrates a typical neutrophil region, while the FL1-H vs. FL2-H plot of FIG. 21B illustrates a gated region R2 showing cells with a high level of staining on the FL1-H axis, indicating CD66b+ neutrophils. Cell counts from FACS and PD microchip imaging were plotted for each sample, as shown in FIG. 22A, and averaged cell counts from FACS and PD microchip imaging (specifically, averaged from three microchannels of each microchip) were plotted, as shown in FIG. 22B. The plot of FIG. 22A illustrates a linear correlation between the two cell counting methods, with a correlation coefficient of 0.383 (p<0.005), therefore showing that an increase in the concentration of sample injection will result in an overall higher cell count using either method.

Although the results illustrated in FIGS. 22A and 22B show high variances on PD microchip cell count values, these high variances are most likely a result of experimental procedure issues. Such issues include large bubble formations (for example, as a result of CCD heating causing evaporation), cell gradients in the syringes during PD sample injection, and overall microchip noise. However, these problems can be improved upon through CCD live imaging to determine dead volumes in tubing that contains no neutrophils, using ethanol and lens cleaner on the microchip to reduce noise, soaking microchips in higher volumes of PBS to prevent drying and evaporation, consistent CCD cooling to prevent evaporation, optimizing flow rates, etc. Magnetic beads can also be used to decrease the variation of neutrophil concentration in the samples. While streptavidin may be used in place of neutravidin, it was shown that streptavidin-treated microchips provided very low capture efficiencies and manually treated neutravidin microchips provided much higher efficiencies.

The present invention has been described in terms of one or more preferred embodiments, and it should be appreciated that many equivalents, alternatives, variations, and modifications, aside from those expressly stated, are possible and within the scope of the invention.

1. A system for point-of-care (POC) testing of biological samples for biomarkers indicative of a predetermined pathological condition, the system comprising:
a microchip system configured to receive a biological sample secured from a patient and provide colorimetric biomarker feedback indicative of a testing related to the predetermined pathological condition;
a mobile device configured to access a communications network and having a processor configured to:
access a camera configured to acquire color images of the colorimetric biomarker feedback;
determine a color intensity of at least a selected portion of the color image;
correlate the color intensity of the selected portion of the color image with a biomarker concentration; and
generate a report regarding the concentration of the biomarker concentration.

2. The system of claim 1 in which the processor is further configured to correlate the biomarker concentration with a presence of the predetermined pathological condition and indicate at least one of a presence and an absence of the predetermined pathological condition in the report.

3. The system of claim 1 in which the microchip system is configured to perform an enzyme-linked immunosorbent assay specific to the predetermined pathological condition.

4. (canceled)

5. The system of claim 1 in which determining a color intensity is performed by comparing pixel values to a predetermined threshold to identify the at the portion of the color image and determining color pixel values of the selected portion.

6. The system of claim 1 in which determining a color intensity comparing an initial color intensity of a background portion of the color image to a predetermined color intensity of a background portion of a calibration image and includes normalizing the color intensity based on the comparison.

7. (canceled)

8. The system of claim 1 in which the predetermined pathological condition includes ovarian cancer and the biological sample include urine.

9. (canceled)

10. The system of claim 1 in which the microchip is configured to utilize a one of CD4 cells, nuetrophils, a kidney injury molecule 1 biomarker, a brain-derived neurotrophic factor biomarker, a cancer antigen 125 biomarker, and E. coli to provide the colorimetric biomarker feedback.

11. The system of claim 1 further comprising a lighted enclosure configured to enclose the microchip and the mobile device to provide a light-controlled environment for acquiring the color images of the colorimetric biomarker feedback.

12. The system of claim 1 in which the mobile device is one of a cell phone with a built-in camera and a tablet having access to a lensless charge-coupled device.

13. A method for analyzing a biomarker in a biological sample, the steps of the method comprising:
a) loading the biological sample onto a microchip;
b) performing an enzyme-linked immunosorbent assay specific to the biomarker on the microchip;
c) generating a color image of the microchip using one of a mobile device and a lensless charge coupled device;
d) determining a color intensity of a selected portion of the color image;
e) correlating the color intensity with a biomarker concentration using a baseline curve calculation; and
f) reporting the concentration of the biomarker.

14. The method as recited in claim 13 in which the biomarker is human epididymis protein 4

15. The method as recited in claim 13 in which step f) includes displaying the concentration of the biomarker on the mobile device

16. (canceled)

17. The method as recited in claim 13 further comprising transmitting the color image to an additional device, in which steps d) and e) are performed through an application executed by the additional device.

18. The method as recited in claim 13 further comprising comparing the concentration of the biomarker to a threshold concentration and reporting one of a positive physiological condition result and a negative physiological condition result based on the comparison.

19. The method as recited in claim 13 further comprising positioning the microchip within a lighted enclosure prior to performing step c).

20. The method as recited in claim 13 in which step d) is performed by determining color pixel values of the selected portion, and further comprising selecting the selected portion in step d) by determining at least one region in the color image with color pixel values above a pixel threshold value.

21. (canceled)

22. The method as recited in claim 13 further comprising normalizing the color intensity of the selected portion prior to step e) by comparing a color intensity of a background portion of the color image to a color intensity of a background portion of a calibration image.

23. The method as recited in claim 22 further comprising calculating the baseline curve calculation by performing an enzyme-linked immunosorbent assay on a series of calibration sample microchips with known biomarker concentrations, generating calibration images of the calibration sample microchips using the mobile device, determining calibration color intensities of selected portions of the calibration images, and correlating the calibration color intensities with the known biomarker concentrations.

24. (canceled)

25. A portable test system for mobile device camera-based analysis of biomarker concentrations in biological samples applied to a microchip, the system comprising:
an enclosure adapted to receive the microchip, the enclosure including an imaging aperture large enough to allow imaging of the microchip through the imaging aperture using the mobile device camera;
at least one light source adapted to illuminate the microchip; and
a power source adapted to power the at least one light source.

26. The system of claim 25 further comprising a non-transitive computer-readable storage medium having stored thereon a set of instructions that, when accessed by a processor of the mobile device, is configured to cause the processor to:
acquire at least one color image of the microchip arranged within the enclosure;
determine a color intensity of at least a selected portion of the color image;
correlate the color intensity of the selected portion of the color image with a biomarker concentration; and
generate a report regarding the concentration of the biomarker concentration to provide an indication of at least one of a presence and an absence of cancer.
